(19) United States
(12) Patent Application Publication
(10) Pub. No.: US 2024/0374421 A1
(43) Pub. Date: Nov. 14, 2024

(54) NOZZLE FOR A FLUID DELIVERY DEVICE

(71) Applicant: TWENTY TWENTY THERAPEUTICS LLC, South San Francisco, CA (US)

(72) Inventor: Timothy Stowe, South San Francisco, CA (US)

(21) Appl. No.: 18/196,849

(22) Filed: May 12, 2023

(51) Int. Cl. A61F 9/00; A61J 1/14

(52) U.S. Cl. CPC A61F 9/0026; A61J 1/14

(57) ABSTRACT

A non-gravitational fluid delivery device is provided for delivering fluid to an eye of a user. The device has a cartridge including a container and a cartridge head coupled to the container. The container defines an inner container chamber configured to accommodate a fluid. The head includes opposing first and second walls collectively defining both a nozzle and a nozzle opening extending through the nozzle. The first wall is at least partially formed from a deformable material. The second wall is at least partially formed from a rigid material. Fluid is selectively delivered from the fluid delivery device to the eye through the nozzle opening. The nozzle opening extends in a fluid delivery direction between the first and second walls. The nozzle opening is selectively transitioned between opened and closed conditions via selective manipulation of the first wall.

### TECHNICAL FIELD

This disclosure generally relates to a nozzle for a fluid delivery device. This disclosure also generally relates to a non-gravitational fluid delivery device for delivering fluid to an eye of a user.

### BACKGROUND

Non-gravitational fluid delivery devices for the non-gravitational delivery of fluids (e.g., ophthalmic drugs and/or viscous ophthalmic drugs) to a target site of the user (e.g., to the user's eye(s), nose, and/or mouth) are known. For example, U.S. patent application Ser. No. 15/931,482 (hereafter, “the '482 application”), filed 13 May 2020 by Stowe and titled “Non-Gravitational Fluid Delivery Device For Ophthalmic Applications”, the subject matter of which is incorporated by reference in its entirety, discloses example non-gravitational fluid delivery devices. Known non-gravitational fluid delivery devices include nozzles that are typically formed entirely from rigid materials.

### SUMMARY

In an aspect, alone or in combination with any other aspect, a non-gravitational fluid delivery device is configured to deliver fluid to an eye of a user. The device comprises a cartridge including a cartridge container and a cartridge head coupled to the cartridge container. The cartridge container defines an inner container chamber configured to accommodate a fluid. The cartridge head includes opposing first and second walls collectively defining both a nozzle and a nozzle opening extending through the nozzle. The first wall is at least partially formed from a deformable material. The second wall is at least partially formed from a rigid material. The head also includes the nozzle opening through which fluid is selectively delivered from the fluid delivery device to the eye. The nozzle opening extends in a fluid delivery direction between the first and second walls. The nozzle opening is selectively transitioned between opened and closed conditions via selective manipulation of the first wall. The nozzle opening is at least partially open to permit the fluid flow therethrough when in the opened condition. The nozzle opening is at least partially closed to restrict fluid flow therethrough when in the closed condition.

In an aspect, alone or in combination with any other aspect, a nozzle for a fluid delivery device comprises opposing first and second walls. The first wall is at least partially formed from a deformable material. The second wall is at least partially formed from a rigid material. The nozzle also includes a nozzle opening through which fluid is selectively delivered from the fluid delivery device to a target site. The nozzle opening is defined by the first and second nozzle walls and extend in a fluid delivery direction between the first and second walls. The nozzle opening is selectively transitioned between opened and closed conditions via selective manipulation of the first wall. The nozzle opening is at least partially open to permit the fluid flow therethrough when in the opened condition. The nozzle opening is at least partially closed to restrict fluid flow therethrough when in the closed condition.

### BRIEF DESCRIPTION OF THE DRAWINGS

For a better understanding, reference may be made to the accompanying drawings, in which:

FIG. 1 is a perspective front view of a non-gravitational fluid delivery device according to one aspect of the present invention, including a component in an opened condition;

FIG. 2 is a partial perspective front view of an element of the aspect of FIG. 1, including the element in a first configuration;

FIG. 3 is a schematic cross-sectional view of a portion of the element of FIG. 2;

FIG. 4 is a schematic cross-sectional view of the element of FIG. 2 in a second condition;

FIG. 5 is a top view of a portion of the element of FIG. 2 in an example use condition;

FIGS. 6-7 schematically illustrate an example closure sequence for a portion of the element of FIG. 2;

FIG. 8 is a schematic cross-sectional view of a portion of the element of FIG. 2 in an another example configuration;

FIG. 9 is a schematic illustration of an example fluid delivery system including the device of FIG. 1;

FIG. 10 is a perspective front view of the non-gravitational fluid delivery device of FIG. 1, including the component of FIG. 1 in a closed condition.

FIG. 11 is a partial cross-sectional view taken along lines 11-11 of FIG. 10;

FIG. 12 is a flow diagram illustrating an example method of operation for the device of FIG. 1;

FIG. 13 is a schematic cross-sectional view of another example configuration for a portion of the aspect of FIG. 11; and

FIG. 14 is a partial perspective front view of another example configuration of the element of FIG. 2, including the element in an at least partially opened condition.

### DESCRIPTION OF ASPECTS OF THE DISCLOSURE

Unless defined otherwise, all technical and scientific terms used herein have the same meaning as is commonly understood by one of ordinary skill in the art to which the present disclosure pertains.

As used herein, the term “user” can be used interchangeably to refer to an individual who prepares for, assists with, and/or performs the operation of a tool procedure, and/or to an individual who prepares for, assists with, and/or performs a procedure.

As used herein, the singular forms “a,” “an” and “the” can include the plural forms as well, unless the context clearly indicates otherwise. It will be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that when an element is referred to as being “attached” to, “connected” to, “coupled” with, “contacting,” etc., another element, it can be directly attached to, connected to, coupled with or contacting the other element or intervening elements may also be present. In contrast, when an element is referred to as being, for example, “directly attached” to, “directly connected” to, “directly fixed” to or “directly contacting” another element, there are no intervening elements present. It will also be appreciated by those of skill in the art that references to a structure or feature that is disposed “adjacent” another feature may not have portions that overlap or underlie the adjacent feature.

Spatially relative terms, such as “over” and the like, may be used herein for ease of description to describe one element or feature's relationship to another element(s) or feature(s) as illustrated in the Figures. It will be understood that the spatially relative terms can encompass different orientations of a device in use or operation, in addition to the orientation depicted in the Figures. For example, if a device in the Figures is inverted, elements described as “over” other elements or features would then be oriented “under” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or Figures unless specifically indicated otherwise.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual and partial numbers within that range, for example, 1, 2, 3, 4, 5, 5.5 and 6. This applies regardless of the breadth of the range.

The invention comprises, consists of, or consists essentially of the following features, in any combination.

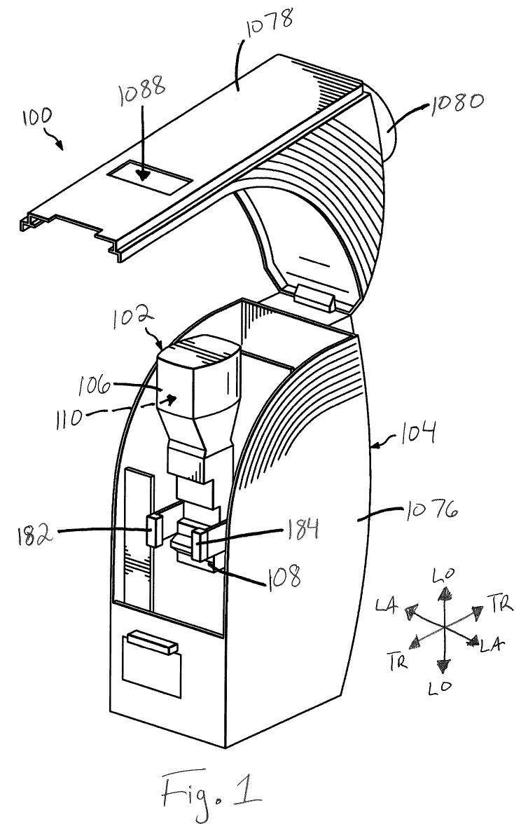

FIG. 1 depicts a non-gravitational fluid delivery device 100 designed in accordance with the present disclosure. The device 100, as largely described herein, is configured for selectively delivering fluid to an eye of a user. However, the device 100 may be configured to deliver fluid to any other portion of the user, such as, for example, the user's nose, mouth, ear(s), limb(s), head, neck, and/or trunk. The device 100 includes a cartridge 102 and an applicator 104 for removably receiving the cartridge 102 therein.

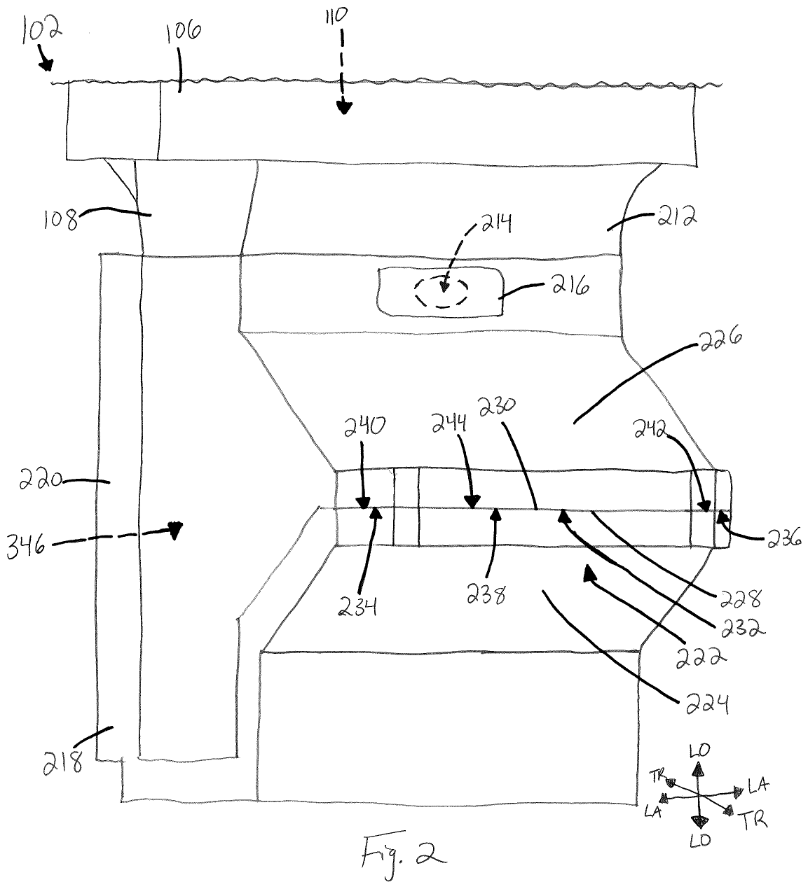

As shown in FIGS. 1-2, the cartridge 102 includes a cartridge container 106 and a cartridge head 108 that is longitudinally adjacent and coupled to the cartridge container 106. The term “longitudinal” is used herein to indicate a substantially vertical direction, in the orientation of FIGS. 1-2, as indicated as “LO” in the Figures. The cartridge container 106 defines an inner container chamber 110 in which a fluid is enclosed. The fluid may be an ophthalmic drug, a viscous ophthalmic drug, any other fluid (viscous or otherwise), or any combination thereof.

As shown in FIGS. 2-3, the cartridge head 108 includes a rigid head portion 212 by which the cartridge head 108 is coupled to the cartridge container 106. The rigid head portion 212 may be at least partially formed from, for example, polyethylene (high density or otherwise), polypropylene, any other rigid material, or any combination thereof. The rigid head portion 212 may include an air entry port 214 formed therein. The air entry port 214 may substantially be, or may substantially be a modified version of, the air entry port(s) of the '482 application. A filter 216 may be positioned over the air entry port 214. The filter 216 may be made from a polypropylene porous material having 0.1 μm to 0.2 μm passages. The filter 216 may be welded, adhered, molded, or otherwise connected directly to the rigid head portion 212.

The cartridge head 108 also includes a deformable head portion 218 welded, adhered, molded, or otherwise connected to the rigid head portion 212. The deformable head portion 218 may be at least partially formed from a deformable material. The deformable material may be an elastomer, such as, but not limited to, silicone, ethylene propylene rubber, any other elastically deformable material, or any combination thereof. The deformable head portion 218 includes a deformable striking wall 220 that is “squeezable” or flexible enough to elastically deflect in response to a striking force being applied thereto.

As shown in FIGS. 2-3, the rigid and deformable head portions 212, 218 collectively form a nozzle 222. In particular, the nozzle 222 is defined by opposing first and second walls 224, 226 that converge toward one another to form the nozzle 222. The first wall 224 is a part of the deformable head portion 218 and, thus, is at least partially formed from the deformable material. The second wall 226, on the other hand, is a part of the rigid head portion 212 and, thus, is at least partially formed from the rigid material.

The first wall 224 includes a first contact surface 228 that faces toward a second contact surface 230 of the second wall 226. A portion first contact surface 228 is fixedly attached to a portion of the second contact surface 230 in such a manner that a nozzle opening 232, which extends in a transverse direction through the nozzle 222, is defined between the first and second contact surfaces 228, 230. The term “transverse” is used herein to indicate a direction substantially perpendicular to the “longitudinal” direction, is shown as a substantially horizontal direction in the orientation of FIG. 3, and is indicated at “TR” in the Figures. The transverse direction, in certain circumstances, may be a fluid delivery direction of the device 100.

As shown in FIG. 2, the first contact surface 228 includes a first end portion 234 laterally separated from a second end portion 236 by a central portion 238. The term “lateral” is used herein to indicate a direction substantially perpendicular to the “longitudinal” and “transverse” directions, is shown as a substantially horizontal direction in the orientation of FIG. 2, and is indicated at “LA” in the Figures. Similarly, the second contact surface 230 includes a first end portion 240 laterally separated from a second end portion 242 by a central portion 244.

The first end portions 234, 240 of the first and second contact surfaces 228, 230 are fixedly attached to one another. The second end portions 236, 242 of the first and second contact surfaces 228, 230 are also fixedly attached to one another. However, the central portions 238, 244 of the first and second contact surfaces 228, 230 are not directly fixed to each other, in many use applications of the device 100. These laterally central portions 238, 244 substantially define the nozzle opening 232, though the first and second end portions 234, 240, 236, 242 may at least partially define the lateral ends of the nozzle opening 232. The nozzle opening 232 thus extends in the fluid delivery direction between the central portions 238, 244 of the first and second contact surfaces 228, 230, and, accordingly, in the fluid delivery direction between the first and second walls 224, 226.

Furthermore, because the central portion 238 of the first contact surface 228 is deformable and not directly fixed to the central portion 244 of the second contact surface 230, the central portion 238 may be selectively manipulated relative to the rigid central portion 244 of the second contact surface 230. Through this manipulation, the nozzle opening 232 may be selectively transitioned between opened and closed conditions. The first wall 224 of the nozzle 222 may be configured such that the nozzle opening 232 is biased to the closed condition. As shown in FIGS. 2-3, when the nozzle opening 232 is in the closed condition, the central portion 238 of the first contact surface 228 contacts the central portion 244 of the second contact surface 230. The contact between the central portions 238, 244 is such that the nozzle opening 232 is substantially (or, in certain configurations, at least partially) closed to restrict fluid flow therethrough.

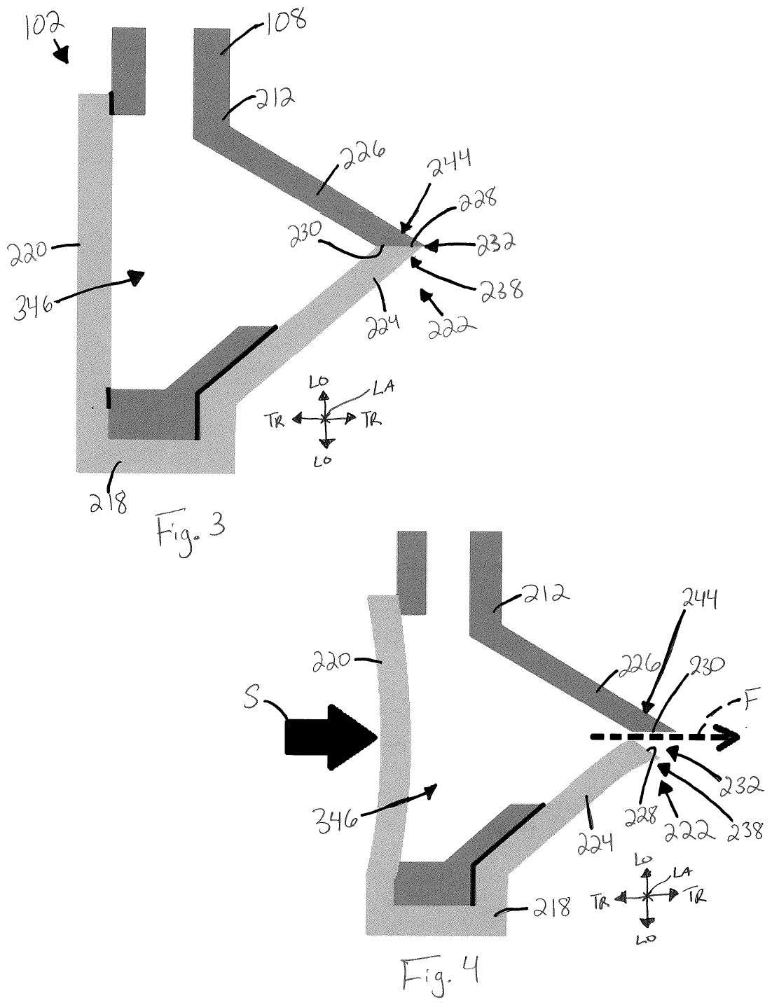

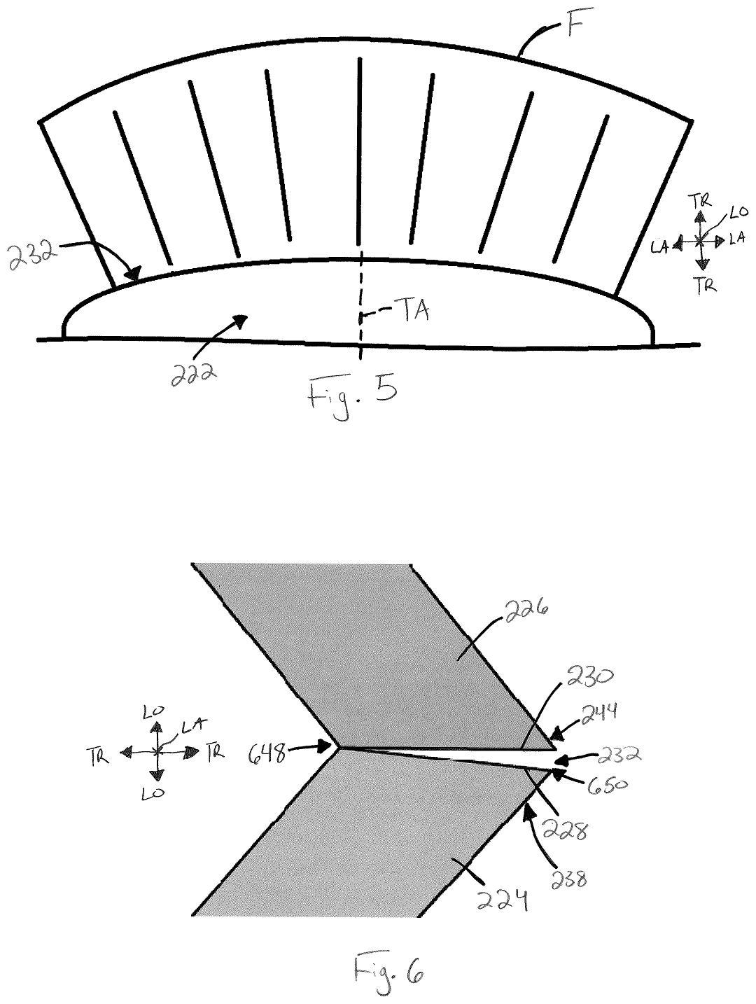

As shown in FIG. 4, when the nozzle opening 232 is in the opened condition, the central portion 238 of the first contact surface 228 is spaced from the central portion 244 of the second contact surface 230. This spacing is such that the nozzle opening 232 is substantially (or, in certain configurations, at least partially) opened to permit the fluid flow therethrough. Therefore, when the nozzle opening 232 is in the opened condition, fluid (schematically represented in FIG. 4 as arrow “F”) may be selectively delivered in the fluid delivery direction from the device 100 to the user's eye.

As shown in FIGS. 3-4, in addition to defining the nozzle 222 and the nozzle opening 232, the rigid and deformable head portions 212, 218 also define an inner head chamber 346 of the cartridge head 108. The striking wall 220 is spaced transversely from the nozzle 222 to locate the head chamber 346 transversely between the striking wall 220 and the nozzle 222. The head chamber 346 is at least temporarily in fluid communication with the container chamber 110 such that fluid selectively flows from the container chamber 110 to the head chamber 346.

During use, a striking force (shown as arrow “S” in FIG. 4) may be selectively applied to the striking wall 220 when the nozzle opening 232 is in the closed position. This striking force responsively causes the striking wall 220 to elastically deflect toward the nozzle 222. Because the nozzle opening 232 may be substantially closed prior to and/or at the beginning of the application of the striking force, fluid pressure in the head chamber 346 increases as the striking wall 220 is deflected. The fluid pressure increases to a level that overcomes the bias of the nozzle/nozzle opening 222/232. In other words, the increased fluid pressure at least partially elastically deforms the first wall 224 (and the central portion 238 of the first contact surface 228 thereof) to at least partially transition the nozzle opening 232 against its internal bias to the opened condition. The applied striking force and deflection of the striking wall 220 also urges the fluid from the head chamber 346 through the opened nozzle opening 232.

Egress of the fluid from the head chamber 346 through the nozzle opening 232 causes the fluid pressure within the head chamber 346 to decrease to a level at which the bias of the first wall 224 (i.e., of the central portion 238 of the first contact surface 228) is able to elastically return the nozzle opening 232 to the closed condition. Once the nozzle opening 232 is at least partially closed, the striking wall 220 may elastically return to its pre-deflected position (see FIG. 3). The return of the striking wall 220 to the pre-deflected position may at least partially generate a vacuum force in the head chamber 346 that draws a predetermined amount of fluid (e.g., a medically predetermined dosage amount of fluid) from the container chamber 110 into the head chamber 346.

The nozzle 222 and the nozzle opening 232 thus function similarly to that of a duckbill valve. In particular, both the nozzle opening 232 and a typical duckbill valve open in response to a predetermined amount of fluid pressure in order to permit fluid to pass therethrough. However, once the fluid pressure decreases or is removed, both the nozzle opening 232 and the duckbill valve return to their closed positions to at least partially prevent backflow and/or containments from entering the system. Typically, known duckbill valves are formed entirely from an elastomer (such as silicone) and, thus, require a relatively high fluidic pressure to open. Therefore, these fully-elastic type duckbill valves generally take several hundred milliseconds to open. Furthermore, the jetting dynamics of a fluid ejecting from this type of duckbill valve may be slowed down because of the significant fluidic pressure and energy losses that result from having to force a fully-elastic duckbill valve open.

The nozzle 222 and the nozzle opening 232 as shown and described herein, however, are designed and operative to at least partially overcome these fully-elastic duckbill valve complications. In particular, in order to provide a faster opening time and faster jetting dynamics than previously possible, the nozzle 222 and the nozzle opening 232 are only partly formed from the deformable material. In other words, the nozzle 222 and the nozzle opening 232 require less force to open, incur less fluidic pressure and energy losses, and provide for a faster operating speed (which includes opening time and fluid dispensing time) as compared to a typical fully-elastic duckbill valve. Therefore, unlike with the known fully-elastic duckbill valve designs, the device 100 is able to open the nozzle opening 232 and deliver the fluid to the user's eye in less than 100 milliseconds, which is fast enough to defeat the blink reflex of the user's eye.

As shown in FIGS. 2 and 5, the first and second walls may be configured such that both the nozzle 222 and the nozzle opening 232 are arcuate along a lateral length of the nozzle 222. This arcuate shaping of the nozzle 222 and the nozzle opening 232 encourages the fluid F exiting the nozzle opening 232 to form a more fan-like shape after exiting. That is, fluid F exiting opposing lateral edges of the nozzle opening 232 will exit at an angle that is not perpendicular to a central transverse axis TA of the nozzle opening 232. Therefore, by making the nozzle 222 and the nozzle opening 232 arcuate, the device 100 is able to produce a relatively large fan-like liquid sheet from a relatively small nozzle 222 and cartridge head 108. Furthermore, the nozzle 222 and the nozzle opening 232 being arcuate may encourage the resulting drop footprint to be more highly elliptical or have an eccentric stadium shape rather than more rounded or ovoid in profile, over greater travel distances to the eye.

As shown in FIGS. 6-7, the first and second walls 224, 226 may be configured such that the nozzle opening 232 gradually closes from an inner nozzle side 648 of the nozzle opening 232 toward an outer nozzle side 650 of the nozzle opening 232 as the nozzle opening 232 is selectively transitioned from the opened condition to the closed condition. In particular, the central portions 238, 244 of the first and second contact surfaces 228, 230 may be angled relative to one another such that the nozzle opening 232 gradually closes from the inner nozzle side 648 toward the outer nozzle side 650. The “zipper-like” gradual closure of the nozzle opening 232 may at least partially prevent air entrainment in the nozzle opening 232, and/or may at least partially prevent small fluid residuals from re-entering into the head chamber 346. This functionality may be particularly useful in preservative-free nozzles.

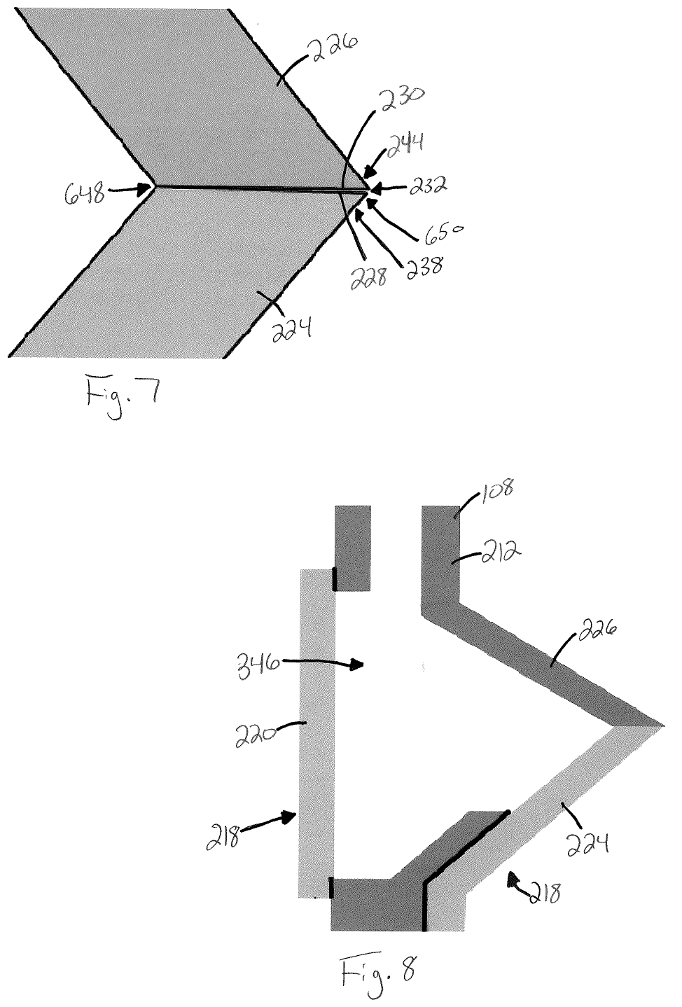

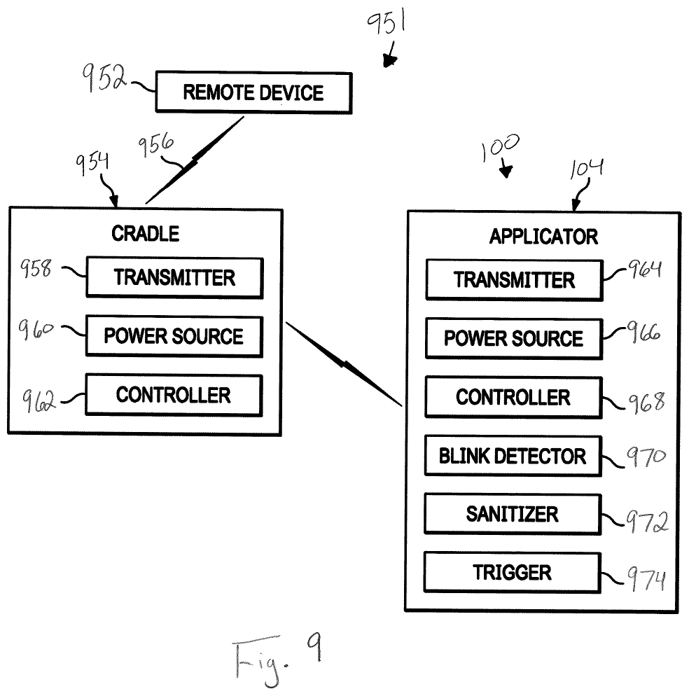

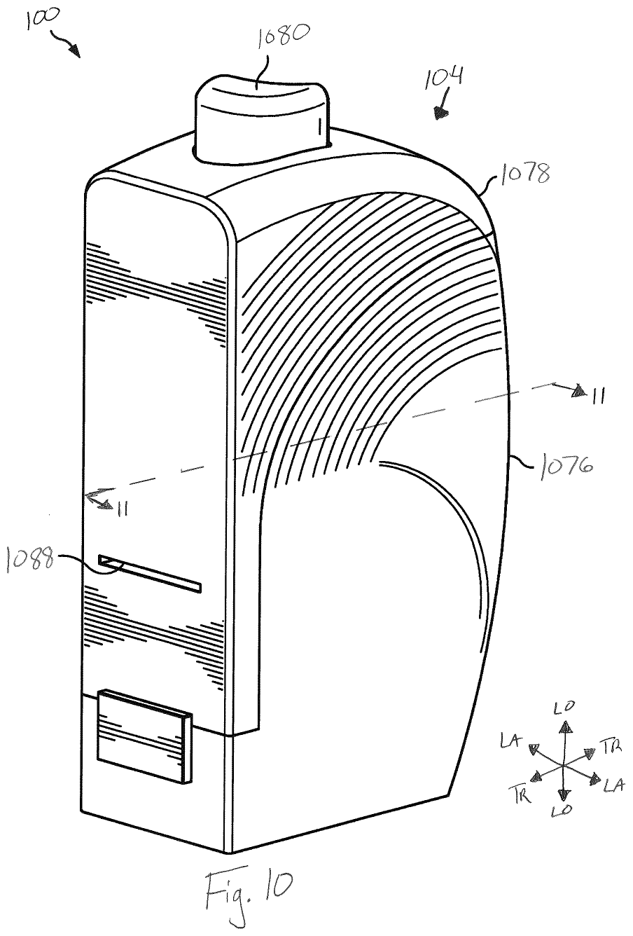

As shown in FIGS. 2-4, the striking wall 220 and the first wall 224 may be integrally formed together as a single monolithic piece that is separate from and subsequently connected to the rigid head portion 212. Alternatively, as shown in FIG. 8, the striking wall 220 and the first wall 224 may instead be separate from one another.

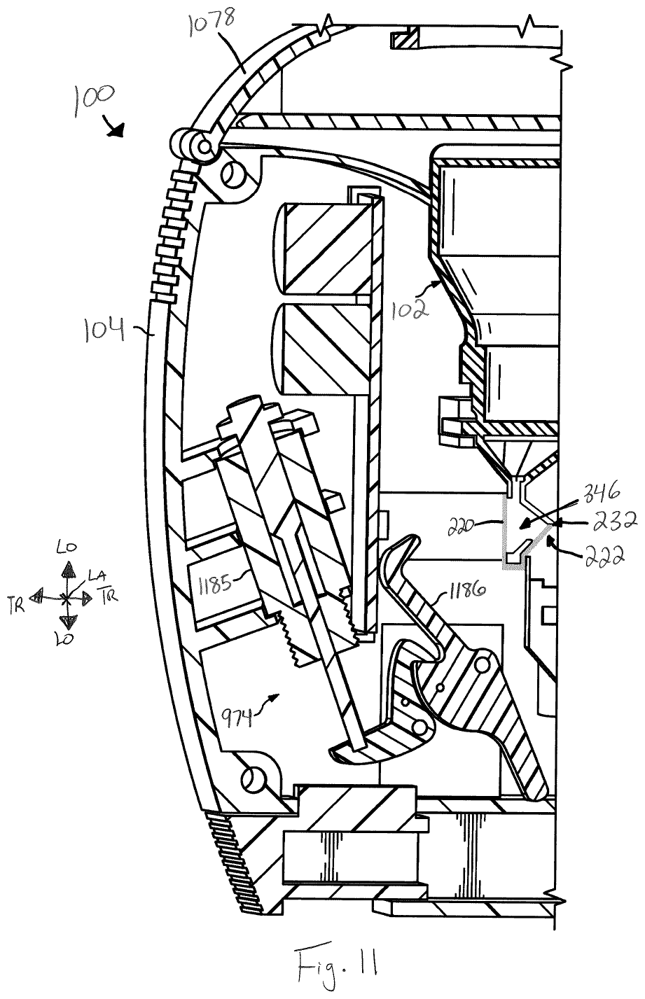

FIG. 9 is a schematic representation of a fluid delivery system 951 having the applicator 104, a remote device 952, and a cradle 954 that may selectively accommodate the applicator 104. Each of the applicator 104, the remote device 952, and the cradle 954 are in communication via a network 956. As illustrated, the cradle 954 includes at least one of a transmitter 958, a power source 960, and a controller 962. The applicator 104 includes at least one of a transmitter 964, a power source 966, a controller 968, a blink detector 970, a sterilizer 972, and a trigger 974. The controller 968 may be operably coupled to at least one of the blink detector 970, the power source 966, the transmitter 964, the sterilizer 972, and the trigger 974.

Referring to FIG. 10, the applicator 104 includes an applicator housing 1076, an applicator cap 1078, and a mechanical activation button 1080. The applicator housing 1076 is sized to accommodate at least one of the cartridge 102, the transmitter 964, the power source 966, the transmitter 964, the sterilizer 972, and the trigger 974. In some configurations, the applicator 104 is an “intelligent” applicator 104 that allows for added user convenience such as, but not limited to, horizontal non-gravitational spray, visual aiming LEDs, blink detection sensors, triggered dispense upon eyelid opening, and/or full cloud connectivity for compliance monitoring. The applicator 104 may be reusably configured replaceable cartridges 102, though it is contemplated that the applicator 104 may instead be configured for one-time use by provision of a non-removable cartridge 102.

The blink detector 970 may substantially be, or may substantially be a modified version of, the blink detector(s) of the '482 application. Therefore, similarly to the blink detector of the '482 application, the blink detector 970 shown in FIG. 1 includes one or more reflective optical proximity infrared sensors 182, 184 (shown here as two sensors 182, 184) to verify proper eye targeting and to detect eye blinks. The controller 968 may be configured to cause the fluid to be dispensed from the device 100 based on signals received from blink detector 970. In particular, the blink detector 970 and the controller 968 may be configured such that the fluid is dispensed from the device 100 a predetermined period of time after a blink event has been detected via the blink detector 970 or upon opening of the eyelids at the tail end of the blink detection event.

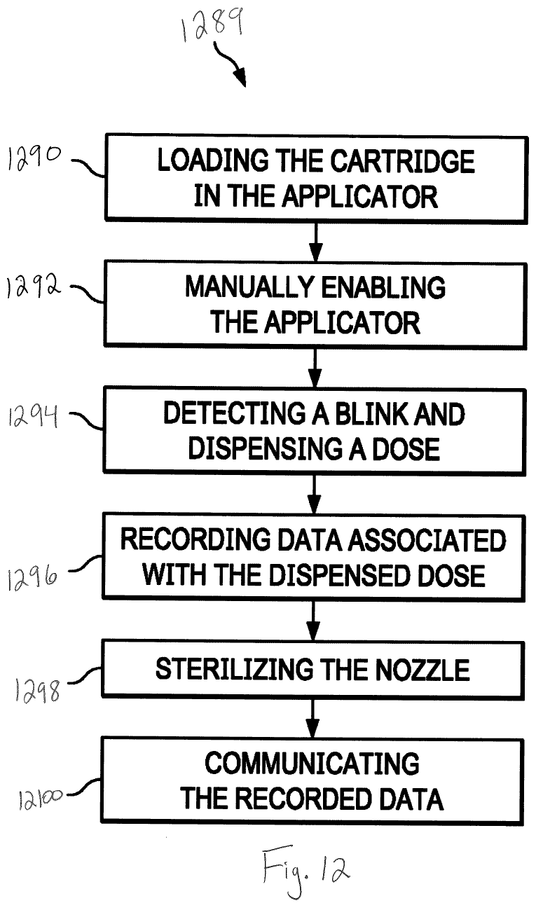

As shown in FIG. 11, the trigger 974 may substantially be, or may substantially be a modified version of, the trigger(s) of the '482 application. In such case, the trigger 974 may be or may include an electromechanical solenoid 1185 that is coupled to an arm or latch trigger 1186 that strikes the striking wall 220. Generally, the trigger 974 is activated by an electrical signal and causes a hard tip object (e.g., portion of solenoid or latch trigger 1186) to apply a striking force via contact to the striking wall 220 and deform the striking wall 220, which responsively increases the fluid pressure in the head chamber 346 and imparts positive displacement of the fluid in the head chamber 346 through the nozzle 222.

The sterilizer 972 of FIG. 9 may substantially be, or may substantially be a modified version of, the sterilizer(s) of the '482 application. Therefore, the sterilizer 972 may comprise at least one ultraviolet (“UV”) light emitting diode (“LED”) that is positioned relative to the nozzle 222 such that the nozzle 222 is exposed to a predetermined amount of UV light when the UV LED is on. Because constant power to the UV LED may use a substantial amount of energy from the power source, the UV LED may be turned on by the controller 968 for only a few seconds after fluid is ejected from the device 100.

The power source 966 may be a rechargeable battery, such as a small coin cell or LiPo battery. Alternatively, the power source 966 may be a non-rechargeable (“single-use”) battery.

The transmitter 964 of the applicator 104 is in electronic communication with the transmitter 958 of the cradle 954. Electronic communication between the transmitters 954, 964 and/or between the transmitters 954, 964 and the remote device 952 allow for tracking the use of the device 100. The electronic communication and connectivity between the cradle 954, the applicator 104, and/or the remote device 952 may allow for time and date tracking of medications, syncing between different devices that are similar or identical to the device 100, auto-re-ordering of medications, providing battery recharge reminders, providing reminders to the user to take medication, enables doctor/user sharing, improving telemedicine options, and/or tracking treatment compliance. The electronic communication and connectivity between the cradle 954, the applicator 104, and/or the remote device 952 may allow for the applicator 104 to be trained based on historical data. Examples of applicator training may include, but are not limited to, updating algorithms and/or calculations using data regarding scleral baseline proximity reflection, skin reflection, movement off axis and centering signals, blink temporal dynamics, or any combination thereof.

As shown in FIG. 10, the applicator cap 1078 is coupled to, and moveable relative to, the applicator housing 1076. For example, the applicator cap 1078 is selectively movable from an opened position (FIG. 1) to a closed position (FIG. 10). When the applicator cap 1078 is in the opened position, at least a portion of an interior of the applicator housing 1076 may be accessed. For example, the cartridge 102 may be inserted into, and/or removed from, the applicator housing 1076 when the applicator cap 1078 is in the opened position. However, once the applicator cap 1078 is moved to the closed position, insertion to, or removal from the applicator housing 1076 of the cartridge 102 is substantially prevented.

The mechanical activation button 1080 is for selectively waking up the device 100, arming the device 100 for usage, and/or responsively causing fluid to be ejected from the nozzle 222 of the cartridge 102 and expelled from the device 100 through an applicator opening 1088 in the applicator housing 1076. The applicator 104 may also include a dust cover, which extends over or across the applicator opening 1088. The dust cover may substantially be, or may substantially be a modified version of, the dust cover(s) of the '482 application. When the dust cover is provided, the mechanical activation button 1080 may be operably coupled to the dust cover for responsively causing the dust cover transitioned away from the applicator opening 1088.

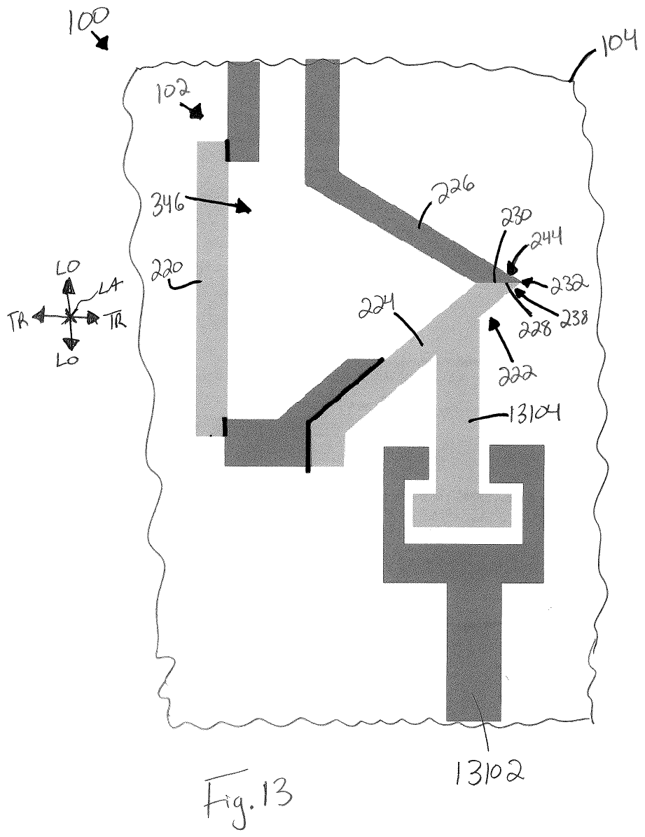

An example sequence 1289 for operating the device 100 is illustrated in FIG. 12. As shown, the sequence 1289 may include at least one of inserting/loading the cartridge 102 into the applicator 104 at step 1290; manually enabling the applicator 104 at step 1292; detecting a blink and dispensing a dose at step 1294; recording data associated with the dispensed dose at step 1296; sterilizing the nozzle 222 at step 1298; and communicating the recorded data via the transmitters 958, 964 at step 12100.

At step 1290, the cartridge 102 is loaded in the applicator 104. Loading the cartridge 102 may include one or both of the insertion of the cartridge 102 into the applicator 104 and the filling of the head chamber 346 with the fluid from the container chamber 110. The head chamber 346 may be fluidically filled via a gravitational force and/or a vacuum force. In some embodiments, the cartridge 102 is disposable. Therefore, for some configurations and under certain situations, inserting the cartridge 102 into the applicator 104 may involve removing a previously used cartridge 102 and inserting a new or different cartridge 102 into the applicator 104.

At step 1292, the applicator 104 is manually enabled with mechanically or electrically loaded energy preparing for a strike to the striking wall 220. The applicator 104 may be manually enabled when the user depresses the mechanical activation button 1080. Although the applicator 104 may be enabled via the depressed mechanical activation button 1080, the device 100 may not be “fired” until the blink detector 970 determines that the applicator 104 is correctly positioned relative to the eye of the user and in response to a detected blink. However, in configurations where the blink detector 970 is omitted from the device 100, fluid may be dispensed from the device 100 upon depression of the mechanical activation button 1080 regardless of the applicator's position and the user's eye/blink status.

The dust cover (when provided) may also be moved away from the applicator opening 1088 upon depression of the mechanical activation button 1080 during step 1292.

At step 1294, a blink is detected and a dose is dispensed. As discussed above, the blink detector 970 determines that the nozzle 22 is aligned with the eye and detects a blink. Upon detecting a blink, the controller 962 sends a signal to the trigger 974 to dispense the dose. The arm or latch trigger 1186 responsively moves from a loaded position (in which the arm or latch trigger 1186 is spaced from the striking wall 220) to the striking position to strike/contact the striking wall 220 and deflect the striking wall 220 toward the nozzle 222. Deflecting the striking wall 220 increases the fluidic pressure in the head chamber 346, which responsively causes the nozzle opening 232 to open and the fluid in the head chamber 346 to be urged/forced through both the nozzle opening 232 and the applicator opening 1088 to the user's eye. After the fluid is dispensed, the fluidic pressure in the head chamber 346 is at least partially alleviated such that the nozzle opening 232 is permitted to return to the closed condition. Also, the striking wall 220 is released from its deflected state and permitted to transition back to an undeflected state after the fluid is dispensed. At least a relatively small vacuum force may be generated in the head chamber 346 as the striking wall 220 transitions back to the undeflected state. Because the nozzle opening 232 is closed, air is substantially prevented from being drawn through the nozzle opening 232 and into the head chamber 346 by the vacuum force. Instead, the vacuum force at least partially causes fluid to be drawn from the container chamber 110 into the head chamber 346.

At the step 1296, the controller 962 records data associated with the dispensed dose. The controller 962 may record data detected by the blink detector 970 and data detected or generated by the trigger 974. As such, the controller 962 may detect and record the timing of each dose being dispensed. Moreover, the controller 962 may detect and record a blink speed of the user.

At step 1298, the sterilizer 972 sterilizes the nozzle 222. For example, in response to a fluid dispense event being detected by the controller 962, the controller 962 may activate the sterilizer 972 for a predetermined period of time to sterilize a portion of the nozzle 222. If a dust cover is provided, the sterilization of the nozzle 222 may occur after the dust cover has returned to a position in which the dust cover substantially covers the applicator opening 1088.

At the step 12100, the recorded data may be communicated via the transmitters 958, 964. For example, the recorded data may be electronically sent to the transmitter 958 and/or the remote device 952. Alternatively or additionally, the data may be transmitted from the transmitter 958 of the cradle 954 to the transmitter 964 of the applicator 104. The recorded data may be stored in the controller 962 of the cradle 954. However, the recorded data may also be stored or received by the remote device 952 via the network 956. The controller 968 of the applicator 104 may upload and update the recorded data to a cloud-based database via the controller 962 of the cradle 954. This recorded data can be used to update, customize, and generate predictive models to refine dry eye management over the course of hours to days. The models may include a variety of factors including historical, current, and expected or predicted external factors, which can be used to generate predictive models. Alternatively, the recorded data could be stored onboard the device 100 as desired, potentially for later retrieval and/or access in any suitable manner.

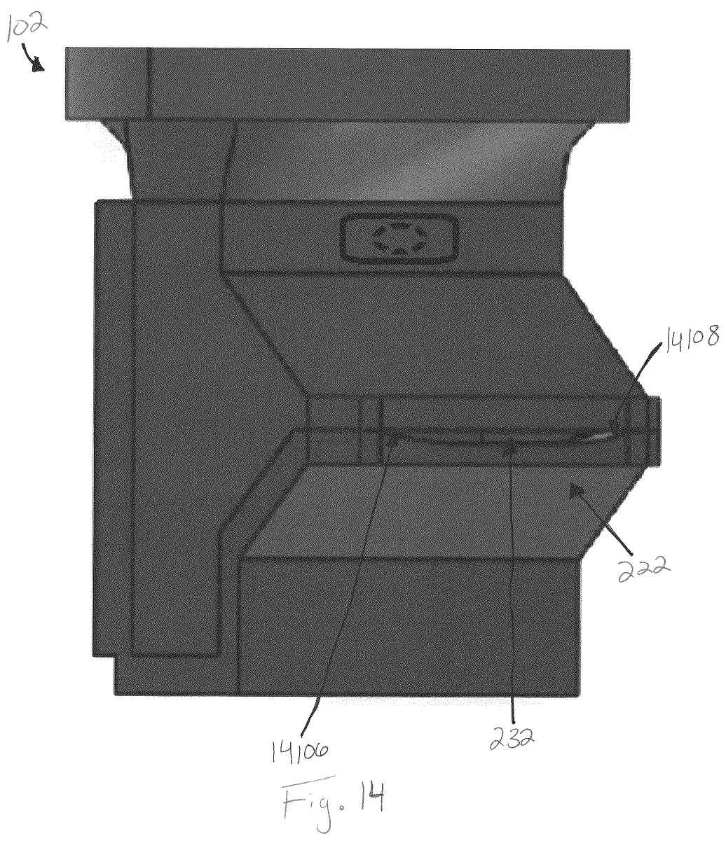

Instead of opening only in response to the fluidic pressure in the head chamber 346, the nozzle opening 232 may be configured to be at least partially opened via a nozzle actuator 13102 of the applicator 104. As shown in FIG. 13, the nozzle actuator 13102 is selectively or substantially permanently attached to the first wall 224. In particular, the nozzle actuator 13102 may be selectively or substantially permanently attached to a projection 13104 that extends from the first wall 224. As shown in FIG. 13, the projection 13104 is formed integrally with the first wall 224 as a single monolithic piece, though the projection 13104 may be formed separately and subsequently attached to the first wall 224. Alternatively, the nozzle actuator 13102 may be may be selectively or substantially permanently attached directly to the first wall 224.

The nozzle actuator 13102 is selectively movable between a first position (FIG. 13) and a second position. As shown in FIG. 13, the nozzle opening 232 is in the closed condition when the nozzle actuator 13102 is in the first position. During a dispensing operation, the nozzle actuator 13102 is moved from the first position to the second position. As the nozzle actuator 13102 moves to the second position, the nozzle actuator manipulates the first wall 224 via the projection 13104 so that the nozzle opening 232 is moved toward the opened condition. For example, the nozzle actuator 13102 may at least partially urge or pull the central portion 238 of the first contact surface 228 to the opened condition via the projection 13104 as the nozzle actuator 13102 transitions to the second position. Therefore, the nozzle opening 232 is in the opened condition when the nozzle actuator 13102 is in the second position.

Having the nozzle actuator 13102 open the nozzle opening 232 either on its own or in conjunction with the fluid pressure in the head chamber 346 at least partially reduces the amount of fluidic pressure needed to open the nozzle opening 232 and, thus, further reduces the fluidic pressure and energy losses in the device 100. Furthermore, the nozzle actuator 13102 at least partially urging the nozzle opening 232 toward the opened condition may be useful when residual liquid in the nozzle opening 232 would have otherwise at least partially prevented the nozzle opening 232 from opening as desired.

The nozzle actuator 13102 may also be configured to manipulate the first wall 224 to at least partially help close the nozzle opening 232. In other words, the nozzle opening 232 may be selectively moved from the second position to the first position after the fluid is dispensed. As the nozzle actuator 13102 moves to the first position, the nozzle actuator 13102 manipulates the first wall 224 via the projection 13104 so that the nozzle opening 232 is moved toward the closed condition. For example, the nozzle actuator 13102 may at least partially urge or push the central portion 238 of the first contact surface 228 to the closed condition via the projection 13104 as the nozzle actuator 13102 transitions to the first position.

The nozzle actuator 13102 being able to close the nozzle opening 232 is particularly useful when the nozzle opening 232 is biased to the opened configuration. FIG. 14 depicts the nozzle 222 and the nozzle opening 232 being configured to be biased to the opened condition by having gradual parabolic tails 14106, 14108 at each lateral end of the nozzle opening 232. Because the nozzle opening 232 is biased to the opened condition, the nozzle actuator 13102, when in the second position, may selectively impart a closing force directly or indirectly onto the first wall 224. The closing force at least partially urges the central portion 238 of the first contact surface 228 into contact with the central portion 244 of the second contact surface 230 to close the nozzle opening 232 against its bias. When desired, the nozzle actuator 13102 may be moved to the first position to at least partially cease the application of the closing force. The nozzle opening 232 is permitted to elastic return back to the opened condition upon removal of the closing force. The nozzle actuator 13102 moving to the first position may also at least partially urge or pull the central portion 238 of the first contact surface 228 toward the opened condition in order to at least assist with the nozzle opening's return to the opened condition.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, [certain of] the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified--a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages may be obtained from a study of the drawings, the disclosure, and the appended claims.

I claim:

1. A non-gravitational fluid delivery device for delivering fluid to an eye of a user, the device comprising:
a cartridge including a container and a cartridge head coupled to the cartridge container, the cartridge container defining an inner container chamber configured to accommodate a fluid, the cartridge head including
opposing first and second walls collectively defining both a nozzle and a nozzle opening extending through the nozzle, the first wall being at least partially formed from a deformable material, the second wall being at least partially formed from a rigid material, and
the nozzle opening through which fluid is selectively delivered from the fluid delivery device to the eye, the nozzle opening extending in a fluid delivery direction between the first and second walls, the nozzle opening being selectively transitioned between opened and closed conditions via selective manipulation of the first wall, the nozzle opening being at least partially open to permit the fluid flow therethrough when in the opened condition, the nozzle opening being at least partially closed to restrict fluid flow therethrough when in the closed condition.

2. The device of claim 1, wherein the cartridge head includes a deformable striking wall that is spaced transversely from the nozzle to form an inner head chamber, the head chamber being in fluid communication with container chamber such that the fluid selectively flows from the container chamber to the head chamber.

3. The device of claim 2, further comprising an applicator configured to accommodate the cartridge, the applicator including a trigger movable between a loaded position and a striking position, the trigger being spaced from the striking wall when in the loaded position, the trigger contacting and deflecting the striking wall toward the nozzle when in the striking position, selective deflection of the striking wall urging the fluid from the head chamber through the nozzle opening.

4. The device of claim 3, wherein a fluid pressure in the head chamber increases as the striking wall is deflected, the increased fluid pressure at least partially deforming the first wall to at least partially transition the nozzle opening from the closed condition to the opened condition.

5. The device of claim 2, wherein the cartridge head includes a rigid head portion and a deformable portion, the rigid head portion including the second wall, the deformable head portion being connected to the rigid head portion and including the first wall and the striking wall.

6. The device of claim 5, wherein the first wall and the striking wall are integrally formed together as a single monolithic piece that is separate from and subsequently connected to the rigid head portion.

7. The device of claim 1, wherein the first and second walls are configured such that both the nozzle and the nozzle opening are arcuate along a lateral length of the nozzle.

8. The device of claim 1, wherein the first and second walls are configured such that the nozzle opening gradually closes from an inner side of the nozzle opening toward an outer side of the nozzle opening as the nozzle opening is selectively transitioned from the opened condition to the closed condition.

9. The device of claim 1, further comprising an applicator configured to accommodate the cartridge, the applicator including a nozzle actuator configured to selectively manipulate the first wall, the nozzle actuator being selectively movable between a first position and a second position, the nozzle opening being in the closed condition when the nozzle actuator is in the first position, the nozzle opening being in the opened condition when the nozzle actuator is in the second position.

10. The device of claim 1, wherein the first wall includes a first contact surface facing toward a second contact surface of the second wall, a portion first contact surface being fixedly attached to a portion of the second contact surface in such a manner that the nozzle opening is defined between the first and second contact surfaces.

11. The device of claim 10, wherein a laterally central portion of the first contact surface contacts the second contact surface when the nozzle opening is in the closed condition, the central portion of the first contact surface being spaced from the second contact surface when the nozzle opening is in the opened condition.

12. The device of claim 10, wherein the first contact surface of the first wall includes a first end portion laterally separated from a second end portion by a central portion, and the second contact surface of the second wall includes a first end portion laterally separated from a second end portion by a central portion,
wherein the first end portions of the first and second contact surfaces are fixedly attached to one another, and the second end portions of the second of the first and second contact surfaces are fixedly attached to one another,
wherein when nozzle opening is in the closed condition, the central portions of the first and second contact surfaces contact one another, and
wherein when nozzle opening is in the opened condition, the central portions of the first and second contact surfaces are spaced from one another.

13. The device of claim 12, wherein the central portions of the first and second contact surfaces are angled relative to one another such that the nozzle opening gradually closes from an inner side of the nozzle opening toward an outer side of the nozzle opening as the nozzle opening is selectively transitioned from the opened condition to the closed condition.

14. A nozzle for a fluid delivery device, the nozzle comprising:
opposing first and second walls, the first wall being at least partially formed from a deformable material, the second wall being at least partially formed from a rigid material, and
a nozzle opening through which fluid is selectively delivered from the fluid delivery device to a target site, the nozzle opening being defined by the first and second nozzle walls and extending in a fluid delivery direction between the first and second walls, the nozzle opening being selectively transitioned between opened and closed conditions via selective manipulation of the first wall, the nozzle opening being at least partially open to permit the fluid flow therethrough when in the opened condition, the nozzle opening being at least partially closed to restrict fluid flow therethrough when in the closed condition.

15. The device of claim 14, wherein a selective increase in fluid pressure in the fluid delivery device deforms the first wall to at least partially transition the nozzle opening from the closed condition to the opened condition.

16. The device of claim 14, wherein the first and second walls are configured such that both the nozzle and the nozzle opening are arcuate along a lateral length of the nozzle.

17. The device of claim 14, wherein the first and second walls are configured such that the nozzle opening gradually closes from an inner side of the nozzle opening toward an outer side of the nozzle opening as the nozzle opening is selectively transitioned from the opened condition to the closed condition.

18. The device of claim 14, wherein the first wall includes a first contact surface facing toward a second contact surface of the second wall, a portion first contact surface being fixedly attached to a portion of the second contact surface in such a manner that the nozzle opening is defined between the first and second contact surfaces.

19. The device of claim 18, wherein a laterally central portion of the first contact surface contacts the second contact surface when the nozzle opening is in the closed condition, the central portion of the first contact surface being spaced from the second contact surface when the nozzle opening is in the opened condition.

20. The device of claim 18, wherein the first contact surface of the first wall includes a first end portion laterally separated from a second end portion by a central portion, and the second contact surface of the second wall includes a first end portion laterally separated from a second end portion by a central portion,
wherein the first end portions of the first and second contact surfaces are fixedly attached to one another, and the second end portions of the second of the first and second contact surfaces are fixedly attached to one another,
wherein when nozzle opening is in the closed condition, the central portions of the first and second contact surfaces contact another, and
wherein when nozzle opening is in the opened condition, the central portions of the first and second contact surfaces are spaced from one another.
